(19) United States
(12) Patent Application Publication
(10) Pub. No.: US 2016/0163175 A1
(43) Pub. Date: Jun. 9, 2016

(54) SAFETY AND FUEL LEVEL COMMUNICATION SYSTEM

(71) Applicant: Bond Manufacturing Co., Inc., Antioch, CA (US)

(72) Inventor: James C. Jenkins, Marco Island, FL (US)

(73) Assignee: Bond Manufacturing Co., Inc., Antioch, CA (US)

(21) Appl. No.: 14/558,747

(22) Filed: Dec. 3, 2014

(51) Int. Cl. G08B 21/16; G01G 19/414; G08C 17/02; G01N 33/22; G08B 21/18; G01N 33/00; G01F 23/20

(52) U.S. Cl. CPC G08B 21/16; G01N 33/0027; G01G 19/414; G01F 23/20; G01N 33/225; G08B 21/182; G08C 17/02

(57) ABSTRACT

Provided herein are, among other things, an electronic pad comprising a weight sensor, a gas sensor, a processor and a wireless transmitter. Also provided herein is a safety and fuel level communication system comprising the electronic pad disclosed herein and a wireless transceiver. The electronic pad disclosed herein or the system disclosed herein can automatically provide a low gas level alert when the gas amount remained in a gas container on the electronic pad is below a pre-determined level and/or a gas leak alert when a gas leak is detected. Also provided herein is a method of providing a low gas level alert and a gas leak alert.

### FIELD OF THE INVENTION

Provided herein is an electronic pad comprising a weight sensor, a gas sensor, a processor and a wireless transmitter. Also provided herein is a safety and fuel level communication system comprising the electronic pad disclosed herein and a wireless transceiver. The electronic pad disclosed herein or the system disclosed herein can automatically provide a gas leak alert when a gas leak is detected, and a low gas level alert when the remaining gas amount of a gas in a gas container on the electronic pad is below a pre-determined level.

### BACKGROUND OF THE INVENTION

Liquefied petroleum gas (aka LPG or LP gas), such as propane, butane, propylene, butadiene, butylene, isobutylene and mixtures thereof, can be used as a fuel for heating appliances, cooking equipment, and vehicles. LPG is also widely used in North America, China, Australia and Europe for domestic cooking and outdoor grilling. LPG is generally stored in gas containers such as propane tanks and propane cylinders.

There are two problems associated with using LPG stored in a gas container. First, because LPG is highly combustible, there is always a risk of fire and explosion if the LPG inside the gas container leaks out. Explosion may occur if the mixture of LPG and air is within the explosive limits and there is an ignition source. Second, there is a risk of running out of the LPG inside the gas container during the LPG is being consumed.

Regarding the risk of fire and explosion, the most common method to minimize the risk is to monitor the occurrence of a leaking event and to provide a timely warning or alert before any tragedy occurs. In general, a gas detector can be used to detect a gas leak and provide an alarm to people in the area where the leak is occurring, giving them the opportunity to leave or shut down the gas container.

Regarding the risk of running out of the LPG, the most common method to avoid the risk is to keep track of how much LPG is left in the gas container. One way to keep track of how much LPG is left, a propane level gauge, propane tank gauge, float gauge, face gauge, or magnetic level gauge can be used for indicating the approximate level of LPG inside the gas container. Another way is an inconvenient 3-step weighing method comprising: (1) weighing an empty gas container on a scale; (2) weighing a second gas container with the LPG to find the total weight; and (3) subtracting the weight of the empty gas container from the total weight to find out the weight of the LPG in the second gas container.

Currently, there is no device or system that can automatically detect a gas leak and keep track of how much LPG is left in a gas container. Therefore, there is a need for a device or system that can both detect a gas leak as well as keep track of how much LPG is left in the gas container easily and automatically. There is also a need for a device or system that can automatically provide a gas leak alert when a gas leak is detected, and a low gas level alert when the remaining gas amount in the gas container is below a pre-determined level.

### SUMMARY OF THE INVENTION

Provided herein are devices and systems that can automatically provide a gas leak alert and a low gas level alert.

In one aspect, provided herein is an electronic pad comprising:

a) a weight sensor, which is arranged to measure a weight of a gas container on the top of the electronic pad, for producing a weight measurement output;

b) a gas sensor, which is arranged to detect a gas, for producing a gas measurement output;

c) a processor for processing the weight measurement output and the gas measurement output to provide weight information and gas information; and

d) a first wireless transmitter for transmitting the weight information and gas information to a wireless receiver.

In some embodiments, the electronic pad disclosed herein further comprises a first alarm for providing an audible signal and/or a visible signal to alert people when the gas is detected.

In certain embodiments, the weight sensor disclosed herein is a force gauge, a hydraulic load cell, a pneumatic load cell, a strain-gauge load cell, or a piezoelectric load cell.

In some embodiments, the gas sensor disclosed herein is an infrared point gas sensor, an ultrasonic gas sensor, an electrochemical gas sensor, or a semiconductor gas sensor. In other embodiments, the gas sensor disclosed herein is for detecting propane, butane, propylene, butadiene, butylene, isobutylene or an odorant therein, or a mixture thereof.

In certain embodiments, the first wireless transmitter disclosed herein comprises a Bluetooth transmitter, a Wi-Fi transmitter, a ZigBee transmitter or a combination thereof.

In some embodiments, the electronic pad disclosed herein further comprises a housing with a top surface adapted to engage the bottom of the gas container. In other embodiments, the top surface comprises a ring or a cup adapted to engage the bottom of the gas container.

In certain embodiments, the gas container disclosed herein is a propane tank or cylinder. In other embodiments, the propane tank or cylinder contains a gas comprising propane, butane, propylene, butadiene, butylene, isobutylene or a mixture thereof. In further embodiments, the capacity of the propane tank or cylinder is from about 15 L to about 100 L.

In another aspect, provided herein is a safety and fuel level communication system comprising a wireless transceiver and the electronic pad disclosed herein. In certain embodiments, the wireless transceiver comprises:

(a) at least one wireless receiver for receiving the weight information and gas information from the first wireless transmitter of the electronic pad; and

(b) a second wireless transmitter for transmitting the weight information and gas information to at least one mobile device configured to receive the weight information and gas information,

wherein the at least one wireless receiver and the second wireless transmitter share or does not share a common circuit.

In some embodiments, the at least one wireless receiver comprises a Wi-Fi receiver and a Bluetooth receiver. In some embodiments, the second wireless transmitter comprises a Wi-Fi transmitter, a Bluetooth transmitter, a ZigBee transmitter or a combination thereof.

In certain embodiments, the wireless transceiver further comprises a second alarm for providing an audible signal and/or a visible signal to alert people when the gas is detected. In other embodiments, the at least one mobile device is a smartphone, a tablet computer, a wearable computer, a pager or a combination thereof.

In some embodiments, the weight information includes remaining gas amount or level, average usage amount, remaining gas usage time, remaining container capacity, and/or a low gas level alert when the remaining gas amount, remaining gas usage time, or remaining container capacity is below a pre-determined level. In other embodiments, the low gas level alert is a real-time text alert.

In certain embodiments, the gas information includes the presence or absence of a gas and a gas leak alert when the gas is detected.

In another aspect, provided herein is a method of providing a low gas level alert and a gas leak alert, comprising:

(a) providing an electronic pad disclosed herein;

(b) placing a gas container having a gas on the electronic pad;

(c) detecting the leaking of the gas from the gas container by the gas sensor;

(d) detecting the amount of the gas in the gas container by the weight sensor; and

(e) sending out a low gas level alert through the first wireless transmitter when the amount of the gas remained in the gas container is less than a pre-determined level and a gas leak alert when the gas is detected by the gas sensor.

### DESCRIPTION OF THE DRAWINGS

FIG. 1 depicts a perspective view of an embodiment of the electronic pad disclosed herein showing a housing with a top surface having a ring adapted to engage the bottom of a propane tank or cylinder.

FIG. 2 depicts a block diagram showing the internal components of an embodiment of the electronic pad of FIG. 1.

FIG. 3 depicts a perspective view of an embodiment of the wireless transceiver disclosed herein.

FIG. 4 depicts a block diagram showing the internal components of an embodiment of the wireless transceiver of FIG. 3.

FIG. 5 depicts a perspective view of an embodiment of the electronic pad of FIG. 1 adapted to engage the bottom of a propane cylinder.

FIG. 6 depicts a perspective view of an embodiment of the wireless transceiver disclosed herein and a smartphone wherein the wireless transceiver is plugged into an electrical outlet and transmitting information to the smartphone.

### DEFINITIONS

The term “sensor” refers to a device that can detect at least one parameter, such as weight, concentration and the presence or absence of a material, and report the at least one parameter as one or more electrical signals. The one or more electrical signals can be displayed directly or further processed by a processor to provide desirable information.

The term “weight sensor” refers to a sensor that can detect or measure the weight of an object or the gravitational force produced by the weight. Some non-limiting examples of the weight sensor include force gauges and load cells such as hydraulic load cells, pneumatic load cells, strain-gauge load cells, and piezoelectric load cells.

The term “gas sensor” refers to a sensor that can detect the presence or absence of, or measure the amount of at least one gas in an area. The gas sensor can be used to detect a gas leak. Some non-limiting examples of the gas sensor include infrared point gas sensors, ultrasonic gas sensors, electrochemical gas sensor, and semiconductor gas sensors.

The term “processor” or “microprocessor” or “central processing unit” or “CPU” refers to an electronic circuit that can carry out the instructions of a computer program by performing the basic arithmetic, logical, control and input/output operations specified by the instructions.

The term “transmitter” or “wireless transmitter” refers to an electronic device which can produce and transmit an electromagnetic wave in the air. The electromagnetic wave can be a radio wave, a microwave or an infrared. In some embodiments, the transmitter can be a separate piece of electronic device, or an electronic circuit within another device. In further embodiments, the transmitter shares at least a portion of its electronic circuit another device such as a receiver.

The term “receiver” or “wireless receiver” refers to an electronic device that can receive an electromagnetic wave in the air and converts the information carried by electromagnetic wave to a usable form. The electromagnetic wave can be a radio wave, a microwave or an infrared. In some embodiments, the receiver can be a separate piece of electronic device, or an electronic circuit within another device. In further embodiments, the receiver shares at least a portion of its electronic circuit with another device such as a transmitter.

The term “transceiver” or “wireless transceiver” refers to a device comprising both a transmitter and a receiver sharing a single housing.

The term “Bluetooth” refers to an industrial specification for wireless personal area networks (PANs). The Bluetooth specifications are developed and licensed by the Bluetooth Special Interest Group. Generally, Bluetooth provides a way to connect and exchange information between devices such as mobile phones, laptops, PCs, printers, digital cameras, and video game consoles over a secure, globally unlicensed short-range radio frequency.

The term “Bluetooth transmitter” refers to a transmitter or a wireless transmitter that meet the Bluetooth specification.

The term “Bluetooth receiver” refers to a receiver or a wireless receiver that meet the Bluetooth specification.

The term “Wi-Fi” (aka Wifi or WiFi or wifi or wi-fi) refers to an industrial specification for the wireless local area networks (WLAN) based on the IEEE 802.11 standard licensed by the Wi-Fi Alliance. Generally, the branding Wi-Fi-CERTIFIED is tested and certified by the Wi-Fi-Alliance. Wi-Fi includes the generic wireless interface of mobile computing devices, such as laptops in LANs. Some non-limiting common uses of Wi-Fi technology include internet and VoIP phone access, gaming, network connectivity for consumer electronics such as laptops in LANs.

The term “Wi-Fi transmitter” refers to a transmitter or a wireless transmitter that meet the Wi-Fi specification.

The term “Wi-Fi receiver” refers to a receiver or a wireless receiver that meet the Wi-Fi specification.

The term “ZigBee” refers to an industrial specification for wireless personal area network (WPAN) based on the IEEE 802.15.4 standard. IEEE 802.15.4 defines a robust radio (PHY) and medium access control (MAC) layer, and ZigBee defines the network, security and application framework for an IEEE 802.15.4-based system. The ZigBee Alliance tests and certifies ZigBee Compliant Platforms. Zigbee technology may be used for low-data rate wireless applications, such as industrial control, embedded sensing, medical data collection, smoke and intruder warning, building automation and home automation.

The term “ZigBee transmitter” refers to a transmitter or a wireless transmitter that meet the ZigBee specification.

The term “ZigBee receiver” refers to a receiver or a wireless receiver that meet the ZigBee specification.

The term “circuit” or “electronic circuit” refers to a configuration comprising individual electronic components, such as resistors, transistors, capacitors, inductors and diodes, connected by conductive wires or traces through which electric current can flow. In certain embodiments, the circuit is an analog circuit, a digital circuit, or a mixed-signal circuit (i.e., a combination of analog circuit and digital circuit). In some embodiments, the circuit can be constructed of discrete components connected by individual pieces of wire. In other embodiments, the circuit comprises a plurality of interconnections created by a photolithographic process on a laminated substrate (e.g., a printed circuit board or PCB) and the components are soldered to the interconnections to create a finished circuit. In further embodiments, the circuit is an integrated circuit or IC wherein the components and interconnections are formed on the same substrate, typically a semiconductor such as silicon or gallium arsenide.

The term “alarm” or “alarm device” refers to a device that can give an audible, visual, textual, or other form of alarm signal about a problem or condition, such as a gas or a gas leak is detected, or the gas level in a gas container is less than a pre-determined level.

The term “gas container” refers to a pressure vessel for storing a gas at above atmospheric pressure. In some embodiments, the gas is a fuel gas such as liquefied petroleum gas (LPG) and liquefied natural gas (LNG). In certain embodiments, the gas container is for domestic uses. In further embodiments, the gas container is for industrial uses.

The term “liquefied petroleum gas” or “LPG” or “LP gas” refers to is a flammable mixture of hydrocarbon gases prepared by refining petroleum or natural gas. In some embodiments, the LPG is commercial propane or HD-5. In certain embodiments, the LPG meets the ASTM D-1835, which is incorporated herein by reference. In other embodiments, the LPG comprises mainly propane and butane. The LPG may further comprise propylene, butylene, isobutylene, butadiene, or a combination thereof. The LPG may further comprise an odorant such as ethanethiol, thiophane, amyl mercaptan or a combination thereof. In some embodiments, the LPG comprises propane, butane, propylene, butylene, isobutylene, butadiene, or a combination thereof.

The term “propane tank or cylinder” refers to a tank or cylinder designed for storing a fuel gas such as an LPG. The capacity of the propane tank or cylinder can be of any useful size. In some embodiments, the capacity of the propane tank or cylinder is from about 0.01 L to about 5000 L, from about 0.1 L to about 1000 L, from about 0.5 L to about 500 L, from about 1 L to about 100 L, from about 1 L to about 50 L, from about 1 L to about 20 L, from about 15 L to about 100 L, or from about 18.9 L to about 90.9 L. In other embodiments, the capacity of the propane tank or cylinder is about 5 gallons (i.e., about 18.9 L), about 7 gallons (i.e., about 26.5 L), about 9 gallons (i.e., about 34.1 L), about 15 gallons (i.e., about 56.8 L), or about 24 gallons (i.e., about 90.9 L). In further embodiments, the propane tank or cylinder is for domestic uses. In certain embodiments, the propane tank or cylinder is for industrial uses.

The term “mobile device” refers to a small, handheld computing device. In certain embodiments, the mobile device comprises a display screen with touch input and/or a miniature keyboard with a weight less than about 10 pounds (i.e., about 4.54 kg), preferably less than about 5 pounds (i.e., about 2.27 kg), or more preferably less than about 2 pounds (i.e., about 0.91 kg). In some embodiments, the mobile device has an operating system (OS) and can run various types of application software. In certain embodiments, the mobile device is equipped with Wi-Fi, Bluetooth, ZigBee and/or GPS capabilities that can allow connections to the Internet and other Wi-Fi-, Bluetooth-, or ZigBee-capable devices. Some non-limiting examples of the mobile device include smartphones, mobile computers (such as tablet computers and wearable computers), and pagers.

The term “smartphone” refers to a mobile phone having an operating system and computing capability. In some embodiments, the smartphone comprises a touchscreen computer, web browser, Wi-Fi, various types of application software, and 3G or 4G capability. Some non-limiting examples of the smartphone include iPhones and various smartphones running the Android mobile operating system.

The term “tablet computer” or “tablet” refers to a mobile computer with display, circuitry and battery in a single housing. In general, the tablet comprises a touchscreen with finger or stylus gestures substituting for the use of computer mouse and keyboard. In some embodiments, the tablet comprises physical buttons for controlling basic features such as speaker volume and power and ports for network communications and for charging the battery. There are different types of tablet such as slate tablet, mini tablet, phablet, booklet, hybrid tablet, laplet, and convertible tablet. Some non-limiting examples of the tablet include Apple iPad tablets, Samsung Galaxy Tab tablets, Google Nexus tablets, Amazon Kindle Fire mini tablets, Microsoft Surface laplets, and Sony Xperia Z phablets.

The term “wearable computer” or “body-borne computer” or “wearable” refers to a mobile device that is worn by the users. Some non-limiting examples of the wearable computers include smartwatches and head-mounted displays.

The term “smartwatch” refers to a computerized wristwatch which runs mobile applications and/or a mobile operating system. In certain embodiments, the smartwatch has full mobile phone capability, and can make or answer phone calls. Some non-limiting examples of the smartwatch include Apple Watch, Samsung Gear smartwatches, LG G Watch, and Sony SmartWatch.

The term “head-mounted display” or “helmet mounted display” or “HMD” refers to a display device, worn on the head or as part of a helmet, that has a small display optic in front of one (monocular HMD) or each eye (binocular HMD).

The term “pager” or “beeper” refers to a wireless telecommunication device that can receive and display numeric or text messages; or receive and announce voice messages.

The term “remaining gas amount” or “remaining gas level” refers to the amount of a gas remained in a gas container during or after the gas in the container is being used or was partly used. The amount or level can be in volume (e.g., gallon or liter) or weight (e.g., pound or Kg) or percentage of the maximum capacity of the container.

The term “average usage amount” refers to the average amount of a gas used per unit time over a time period of prior use of the gas. The amount can be in volume (e.g., gallon or liter) or weight (e.g., pound or Kg). The time unit can be in second, minute, or hour.

The term “remaining gas usage time” refers to the time to completely deplete a gas remained in a gas container by dividing the remaining gas amount by the average usage.

The term “remaining container capacity” refers to the volume (e.g., gallon or liter) or weight (e.g., pound or Kg) of the gas remained in a gas container.

The term “low gas level alert” refers to an alert of a situation where the amount of a gas remained in a gas container is less than a pre-determined level. In some embodiments, the pre-determined level is based on the remaining gas amount where an alert is activated or triggered when the remaining gas amount is less than about 30%, less than about 25%, less than about 20%, less than about 15%, less than about 10%, less than about 5%, less than about 4%, less than about 3%, less than about 2%, or less than about 1%, based on the maximum amount (volume or weight) of gas that can be stored in the gas container. In other embodiments, the pre-determined level is based on the remaining gas usage time where an alert is activated or triggered when the remaining gas usage time is less than about 5 hours, less than about 4 hours, less than about 3 hours, less than about 2 hours, less than about 1 hour, less than about 30 minutes, less than about 15 minutes, less than about 10 minutes, less than about 5 minutes, or less than about 1 minute. In further embodiments, the pre-determined level is based on the remaining container capacity where an alert is activated or triggered when the remaining container capacity is less than about 30%, less than about 25%, less than about 20%, less than about 15%, less than about 10%, less than about 5%, less than about 4%, less than about 3%, less than about 2%, or less than about 1%, based on the total capacity of the gas container.

The term “gas leak alert” refers to an alert of a situation where a gas or a gas leak is detected by a gas sensor. In certain embodiments, the alert is activated or triggered when the concentration of the gas detected by the gas sensor is above or higher than a pre-determined level. In some embodiments, the pre-determined level is the explosion limit of the gas. In some embodiments, the pre-determined level is the detection limit of the gas sensor.

The term “detection limit” of a gas sensor refers to the lowest quantity of a gas that can be detected by the gas sensor.

### DESCRIPTION OF EMBODIMENTS OF THE INVENTION

Provided herein are devices and systems that can automatically provide a low gas level alert when the amount of a gas remained in a gas container is below a pre-determined level and a gas leak alert when a gas leak is detected.

In one aspect, provided herein is an electronic pad comprising:

e) a weight sensor, which is arranged to measure a weight of a gas container on the top of the electronic pad, for producing a weight measurement output;

f) a gas sensor, which is arranged to detect a gas, for producing a gas measurement output;

g) a processor for processing the weight measurement output and the gas measurement output to provide weight information and gas information; and

h) a first wireless transmitter for transmitting the weight information and gas information to a wireless receiver.

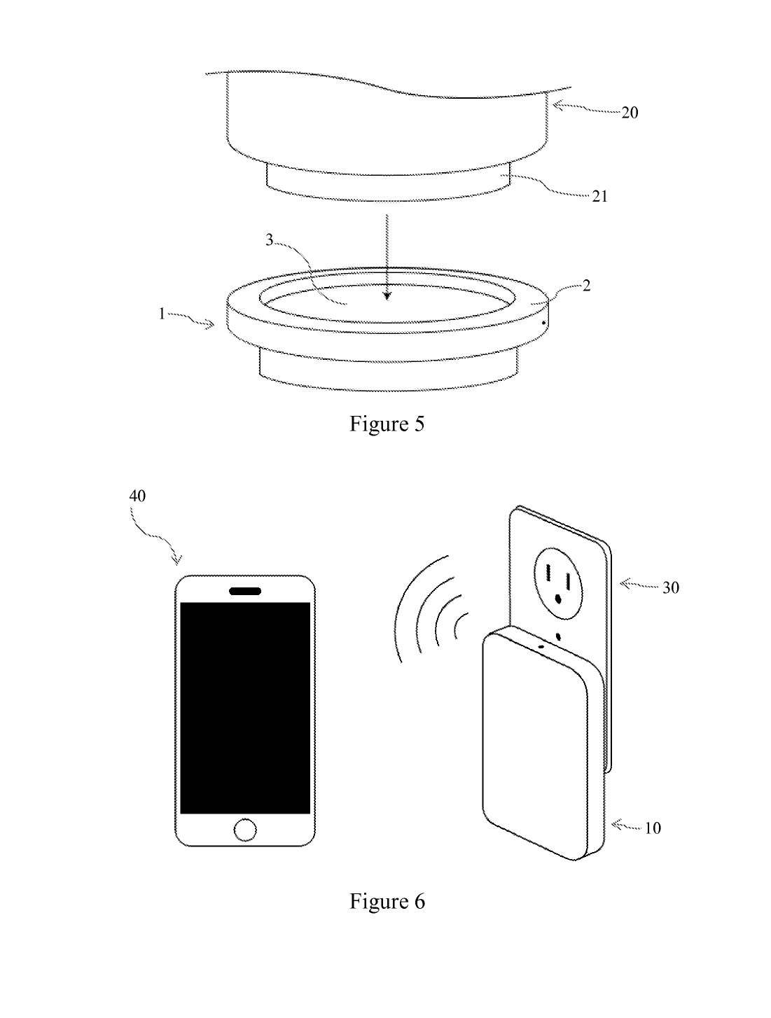

In some embodiments, the electronic pad disclosed herein further comprises a housing with a top surface adapted to engage the bottom of the gas container. In other embodiments, the top surface comprises a ring or a cup adapted to engage the bottom of the gas container. FIG. 1 depicts a perspective view of an embodiment of the electronic pad (1) showing a housing (8) with a top surface (3) having a ring (2) adapted to engage the bottom of a propane tank or cylinder. The housing can be made of a composite, a polymer, or a metal such as stainless steel. The dimension of the housing or the top surface can be any size or shape that is suitable for engaging the propane tank or cylinder. In some embodiments, the electronic pad further comprises a housing and the gas sensor, the weight sensor, the processor, and first wireless transmitter are inside the housing. In further embodiments, the housing further comprises a membrane or an opening that allows the monitored gas to reach the gas sensor inside the housing. In some embodiments, the gas sensor is outside the housing wherein the gas sensor is connected to the processor by an external cable. FIG. 5 depicts a perspective view of the electronic pad of FIG. 1 adapted to engage bottom (21) of propane cylinder (20).

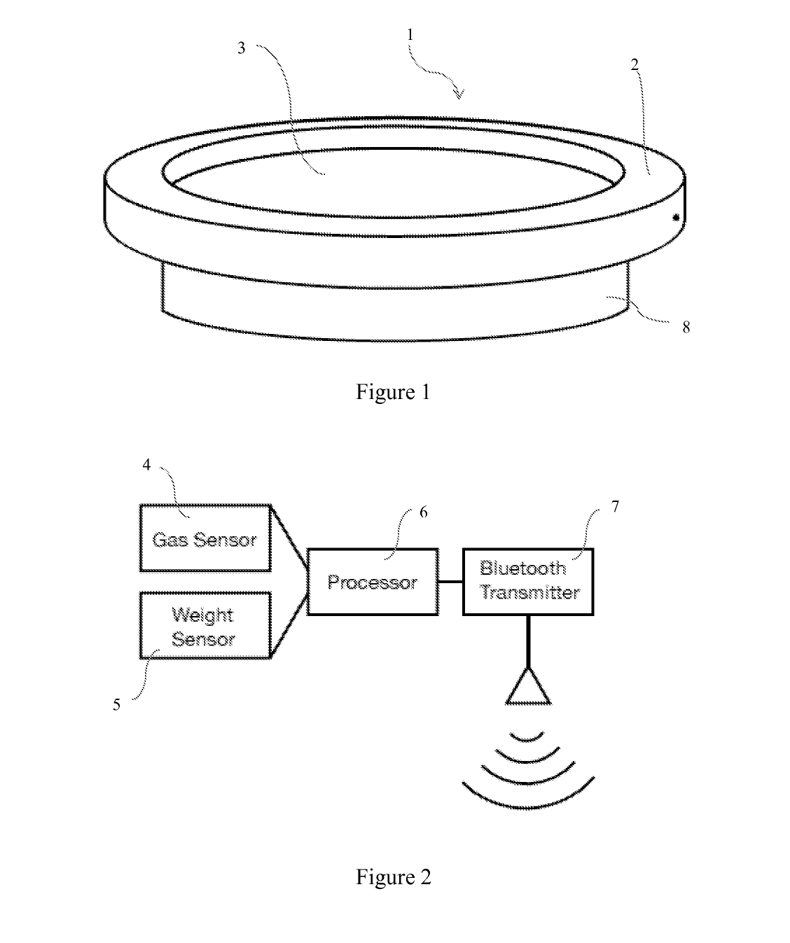

FIG. 2 depicts a block diagram showing the internal components of an embodiment of the electronic pad, including gas sensor (4), weight sensor (5), processor (6), and Bluetooth transmitter (7). FIG. 2 is just one non-limiting embodiment of the electronic pad disclosed herein. Variations and modifications from the embodiment exist. For example, Bluetooth transmitter (7) can be replaced or supplemented with a Wi-Fi transmitter or a ZigBee transmitter.

In some embodiments, the electronic pad may further comprise an indicator for displaying the remaining gas amount or level, remaining gas usage time or remaining container capacity. The indicator may be a part of the electronic pad or an internal device connected to or communicated with the electronic pad with a wire cable or a wireless mean. In certain embodiments, the electronic pad does not comprise an indicator for displaying the remaining gas amount or level, remaining gas usage time or remaining container capacity.

The electronic pad may further comprise one or more additional components such as a wireless receiver for receiving signals or information from an external transmitter, and an alarm for providing an audible signal, a visible signal and/or a real-time text message to alert people when the amount of a gas remained in a gas container is below a pre- determined level, and/or a gas leak alert when a gas leak is detected.

The alarm of the electronic pad disclosed herein can be any device that can give an audible, visual, textual, or other form of alarm signal about a problem or condition. In certain embodiments, the alarm is a speaker alarm. The alarm can be activated by a sensor such as the gas sensors or weight sensors disclosed herein. In some embodiments, the alarm can alert people of the presence of a gas or a gas leak by providing one or more visual signals, one or more audible signals, one or more real-time text alert, or a combination thereof. In other embodiments, the alarm can alert people that the gas level in a gas container is less than a pre-determined level by providing one or more visual signals, one or more audible signals, one or more real-time text alert, or a combination thereof. Some non-limiting examples of the visual signals include a bright red light, a flashing light, a strobe light, or a combination thereof. Some non-limiting examples of the audible signals include high-volume sounds such as a siren, klaxon, pre-recorded verbal warning, or a combination thereof. The audible signals can be generated by a speaker alarm.

The weight sensor disclosed herein can be any sensor that can detect or measure the weight of an object or the gravitational force produced by the weight. The gas sensor can be interfaced with an alarm so that the alarm can provide a warning signal, such as an audible signal, a visible signal and/or real-time text, to alert people when the gas level in a gas container is less than a pre-determined level. In some embodiments, the weight sensor disclosed herein is a force gauge or a load cell, such as hydraulic load cells, pneumatic load cells, strain-gauge load cells, and piezoelectric load cells. In some embodiments, the weight sensor disclosed herein is not a force gauge. In some embodiments, the weight sensor disclosed herein is not a load cell.

The gas sensor disclosed herein can be any sensor that can detect the presence or absence of, or measure the amount of at least one gas in an area. The gas sensor can be interfaced with an alarm so that the alarm can provide a warning signal, such as an audible signal, a visible signal and/or real-time text, to alert people when the monitored gas is detected. The gas sensor can also be interfaced with a control system so that a gas tank can be automatically closed when a gas leak is detected. Some non-limiting examples of suitable gas sensor include infrared point gas sensors, ultrasonic gas sensors, electrochemical gas sensor, and semiconductor gas sensors. In certain embodiments, the gas sensor disclosed herein is for detecting a LPG. In other embodiments, the gas sensor disclosed herein is for detecting an odorant in a LPG. In further embodiments, the gas sensor disclosed herein is for detecting propane, butane, propylene, butadiene, butylene, isobutylene or an odorant therein, or a mixture thereof.

The first wireless transmitter disclosed herein can be any electronic device which can produce and transmit an electromagnetic wave in the air. In certain embodiments, the first wireless transmitter disclosed herein comprises a Bluetooth transmitter, a Wi-Fi transmitter, a ZigBee transmitter or a combination thereof. In some embodiments, the transmitter comprises an antenna and the transmitter generates an alternating current having a radio wave frequency, which is applied to the antenna. When excited by the alternating current, the antenna radiates or transmits the radio wave carrying information. In certain embodiments, the first wireless transmitter can be a separate piece of electronic device, or an electronic circuit within another device. In further embodiments, the first wireless transmitter shares at least a portion of its electronic circuit with another device such as a receiver.

In certain embodiments, the gas container disclosed herein is a propane tank or cylinder. In other embodiments, the propane tank or cylinder contains a gas comprising propane, butane, propylene, butadiene, butylene, isobutylene or a mixture thereof. In further embodiments, the capacity of the propane tank or cylinder is from about 15 L to about 100 L.

In another aspect, provided herein is a safety and fuel level communication system comprising a wireless transceiver and the electronic pad disclosed herein. In certain embodiments, the wireless transceiver comprises:

(a) at least one wireless receiver for receiving the weight information and gas information from the first wireless transmitter of the electronic pad; and

(b) a second wireless transmitter for transmitting the weight information and gas information to at least one mobile device configured to receive the weight information and gas information,

wherein the at least one wireless receiver and the second wireless transmitter share or does not share a common circuit.

The wireless transceiver disclosed herein can be any device that comprises both a transmitter and a receiver sharing a single housing can be used herein as. In some embodiments, the transmitter and the receiver share a common circuit. In other embodiments, the transmitter and the receiver do not share a common circuit. In further embodiments, at least a portion of the transmitter circuit and at least a portion of the receiver circuit are combined or shared.

The at least one wireless receiver disclosed herein can be any electronic device that can receive an electromagnetic wave in the air and converts the information carried by electromagnetic wave to a usable form. In some embodiments, the at least one wireless receiver comprises a Wi-Fi receiver and a Bluetooth receiver. In further embodiments, the at least one wireless receiver comprises a Wi-Fi receiver, a Bluetooth receiver, a ZigBee receiver or a combination thereof. In certain embodiments, the at least one wireless receiver comprises an antenna and the antenna intercepts an electromagnetic wave and converts it into alternating currents which are applied to the receiver, and the receiver extracts desirable information from the alternating currents. The desirable information can be in the form of sound (an audible signal), images (a visual signal) or data (a digital signal). In some embodiments, the at least one wireless receiver can be a separate piece of electronic device, or an electronic circuit within another device. In further embodiments, the receiver shares at least a portion of its electronic circuit with another device such as the second wireless transmitter disclosed herein.

The second wireless transmitter disclosed herein can be any electronic device which can produce and transmit an electromagnetic wave in the air. In certain embodiments, the second wireless transmitter disclosed herein comprises a Bluetooth transmitter, a Wi-Fi transmitter, a ZigBee transmitter or a combination thereof. In other embodiments, the second wireless transmitter can be a separate piece of electronic device, or an electronic circuit within another device. In further embodiments, the second wireless transmitter shares at least a portion of its electronic circuit with another device such as the at least one wireless receiver disclosed herein.

The wireless transceiver may further comprise one or more additional components such as a storage device, e.g., a flash drive or a hard drive, for storing data and/or information; and an alarm, e.g., a speaker alarm.

In certain embodiments, the wireless transceiver further comprises a second alarm for providing an audible signal, a visible signal and/or a real-time text message to alert people when the amount of a gas remained in a gas container is below a pre-determined level, and/or a gas leak alert when a gas leak is detected. Any device that gives an audible, visual, textual, or other form of alarm signal about a problem or condition can be used herein as the second alarm disclosed herein. In certain embodiments, the second alarm is a speaker alarm. The second alarm can be activated by a sensor such as the gas sensors or weight sensors disclosed herein. In some embodiments, the second alarm can alert people of the presence of a gas or a gas leak by providing one or more visual signals, one or more audible signals, one or more real-time text alert, or a combination thereof. In other embodiments, the second alarm can alert people that the gas level in a gas container is less than a pre-determined level by providing one or more visual signals, one or more audible signals, one or more real-time text alert, or a combination thereof. Some non-limiting examples of the visual signals include a bright red light, a flashing light, a strobe light, or a combination thereof. Some non-limiting examples of the audible signals include high-volume sounds such as a siren, klaxon, pre-recorded verbal warning, or a combination thereof. The audible signals can be generated by a speaker alarm.

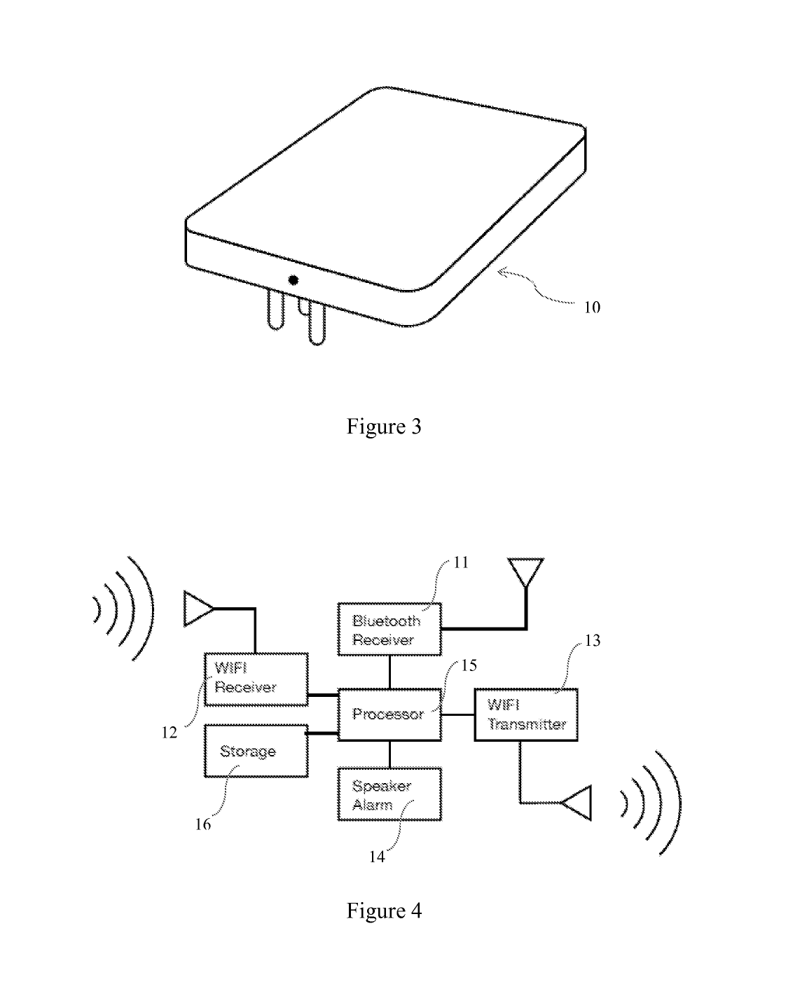

FIG. 3 depicts a perspective view of a wireless transceiver (3). FIG. 4 depicts a block diagram showing the internal components of the wireless transceiver of FIG. 3, including Bluetooth receiver (11), Wi-Fi receiver (12), processor (15), and Wi-Fi transmitter (13), storage or storage device (16), and speaker alarm (14). The Bluetooth receiver (11) is for receiving information from a Bluetooth transmitter such as Bluetooth transmitter (7) of the electronic pad (1). The Wi-Fi receiver (12) is for receiving information from a Wi-Fi transmitter. The information can be stored in storage (16) or processed by processor (15). The information stored in storage (16) or processed by processor (15) can be sent to Wi-Fi transmitter (13) which in turn transmits the information to a mobile device. When the amount of a gas remained in a gas container is below a pre-determined level, and/or a gas leak alert when a gas leak is detected, processor (15) may activated or trigger the speaker alarm (14) to provide an audible alert.

FIG. 6 depicts a perspective view of wireless transceiver (10) of FIG. 3 and smartphone (40) wherein the wireless transceiver is plugged into electrical outlet (30) and transmitting information to smartphone (40).

FIG. 3 or 4 is just one non-limiting embodiment of the wireless transceiver disclosed herein. Variations and modifications from the embodiment exist. For example, a Bluetooth transmitter can be added and/or the speaker alarm can be replaced or supplemented with a visual alarm.

The at least one mobile device can be any small, handheld computing device. In some embodiments, the at least one mobile device is a smartphone, a tablet computer, a wearable computer, a pager or a combination thereof.

In some embodiments, the weight information includes remaining gas amount, average usage amount, remaining gas usage time, remaining container capacity, and/or a low gas level alert when the remaining gas amount, remaining gas usage time, or remaining container capacity is below a pre-determined level. In other embodiments, the low gas level alert is a real-time text alert.

In certain embodiments, the gas information includes the presence or absence of a gas at or near the gas sensor and a gas leak alert when the gas is detected.

In another aspect, provided herein is a method of providing a gas leak alert, comprising:

(a) providing an electronic pad disclosed herein;

(b) placing a gas container having a gas on the electronic pad;

(c) detecting the leaking of the gas from the gas container by the gas sensor; and

(d) sending out a gas leak alert through the first wireless transmitter when the gas is detected by the gas sensor.

In another aspect, provided herein is a method of providing a low gas level alert, comprising:

(a) providing an electronic pad disclosed herein;

(b) placing a gas container having a gas on the electronic pad;

(c) detecting the amount of the gas in the gas container by the weight sensor; and

(d) sending out a low gas level alert through the first wireless transmitter when the amount of the gas remained in the gas container is less than a pre-determined level.

In another aspect, provided herein is a method of providing a low gas level alert and a gas leak alert, comprising:

(a) providing an electronic pad disclosed herein;

(b) placing a gas container having a gas on the electronic pad;

(c) detecting the leaking of the gas from the gas container by the gas sensor;

(d) detecting the amount of the gas in the gas container by the gas sensor; and

(e) sending out a low gas level alert through the first wireless transmitter when the amount of the gas remained in the gas container is less than a pre-determined level and a gas leak alert when the gas is detected by the gas sensor.

In some embodiments, the gas in the methods disclosed herein is a LPG such as propane. In certain embodiments, the gas container in the methods disclosed herein is a propane tank or cylinder. In other embodiments, the methods disclosed herein further comprises receiving the gas leak alert or the low gas level alert by the transceiver disclosed herein and transmitting the gas leak alert or the low gas level alert by the transceiver to one or more mobile devices. In further embodiments, the methods disclosed herein further comprises activating an alarm device disclosed herein when the gas leak alert or the low gas level alert is sent out.

As demonstrated above, embodiments of the invention provide electronic pads or safety and fuel level communication systems that can automatically provide a gas leak alert when a gas leak is detected, and/or a low gas level alert when the remaining gas amount of a gas in a gas container on the electronic pad is below a pre-determined level. While the invention has been described with respect to a limited number of embodiments, the specific features of one embodiment should not be attributed to other embodiments of the invention. No single embodiment is representative of all aspects of the invention. Variations and modifications from the described embodiments exist. The appended claims intend to cover all such variations and modifications as falling within the scope of the invention.

All publications and patent applications mentioned in this specification are herein incorporated by reference to the same extent as if each individual publication or patent application was specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An electronic pad comprising:
a) a weight sensor, which is arranged to measure a weight of a gas container on the top of the electronic pad, for producing a weight measurement output;
b) a gas sensor, which is arranged to detect a gas, for producing a gas measurement output;
c) a processor for processing the weight measurement output and the gas measurement output to provide weight information and gas information; and
d) a first wireless transmitter for transmitting the weight information and gas information to a wireless receiver.

2. The electronic pad of claim 1 further comprising a first alarm for providing an audible signal and/or a visible signal to alert people when the gas is detected.

3. The electronic pad of claim 1, wherein the weight sensor is a force gauge, a hydraulic load cell, a pneumatic load cell, a strain-gauge load cell, or a piezoelectric load cell.

4. The electronic pad of claim 1, wherein the gas sensor is an infrared point gas sensor, an ultrasonic gas sensor, an electrochemical gas sensor, or a semiconductor gas sensor.

5. The electronic pad of claim 1, wherein the gas sensor is for detecting propane, butane, propylene, butadiene, butylene, isobutylene or an odorant therein, or a mixture thereof.

6. The electronic pad of claim 1, wherein the first wireless transmitter comprises a Bluetooth transmitter, a Wi-Fi transmitter, a ZigBee transmitter or a combination thereof.

7. The electronic pad of claim 1, wherein the electronic pad further comprises a housing with a top surface adapted to engage the bottom of the gas container.

8. The electronic pad of claim 7, wherein the top surface comprises a ring or a cup adapted to engage the bottom of the gas container.

9. The electronic pad of claim 8, wherein the gas container is a propane tank or cylinder.

10. The electronic pad of claim 9, wherein the capacity of the propane tank or cylinder is from about 15 L to about 100 L.

11. The electronic pad of claim 1, wherein the weight information includes remaining gas amount, average usage amount, remaining gas usage time, remaining container capacity, and/or a low gas level alert when the remaining gas amount, remaining gas usage time, or remaining container capacity is below a pre-determined level.

12. The electronic pad of claim 1, wherein the gas information includes the presence or absence of a gas and a gas leak alert when the gas is detected.

13. A safety and fuel level communication system comprising a wireless transceiver and the electronic pad of claim 1, wherein the wireless transceiver comprises:
(a) at least one wireless receiver for receiving the weight information and gas information from the first wireless transmitter of the electronic pad; and
(b) a second wireless transmitter for transmitting the weight information and gas information to at least one mobile device configured to receive the weight information and gas information,
wherein the at least one wireless receiver and the second wireless transmitter share or does not share a common circuit.

14. The system of claim 13, wherein the at least one wireless receiver comprises a Wi-Fi receiver and a Bluetooth receiver.

15. The system of claim 13, wherein the second wireless transmitter comprises a Wi-Fi transmitter, a Bluetooth transmitter, a ZigBee transmitter or a combination thereof.

16. The system of claim 13, wherein the wireless transceiver further comprises a second alarm for providing an audible signal and/or a visible signal to alert people when the gas is detected.

17. The system of claim 13, wherein the at least one mobile device is a smartphone, a tablet computer, a wearable computer, a pager or a combination thereof.

18. The system of claim 13, wherein the weight information includes remaining gas amount, average usage amount, remaining gas usage time, remaining container capacity, and/or a low gas level alert when the remaining gas amount, remaining gas usage time, or remaining container capacity is below a pre-determined level.

19. The system of claim 13, wherein the gas information includes the presence or absence of a gas and a gas leak alert when the gas is detected.

20. A method of providing a low gas level alert and a gas leak alert, comprising the steps of:
(a) providing an electronic pad of claim 1;
(b) placing a gas container having a gas on the electronic pad;
(c) detecting the leaking of the gas from the gas container by the gas sensor;
(d) detecting the amount of the gas in the gas container by the weight sensor; and
(e) sending out, through the first wireless transmitter, a low gas level alert when the amount of the gas remained in the gas container is less than a pre-determined level; and a gas leak alert when the gas is detected by the gas sensor.
